(19) United States
(12) Patent Application Publication
(10) Pub. No.: US 2010/0241069 A1
(43) Pub. Date: Sep. 23, 2010

(54) OSTIAL LESION STENT DELIVERY SYSTEM

(75) Inventor: Thomas Ray Hatten, Los Altos, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS INC., Santa Clara, CA (US)

(21) Appl. No.: 12/407,672

(22) Filed: Mar. 19, 2009

(51) Int. Cl. A61M 29/00; A61F 2/06

(52) U.S. Cl. 604/96.01; 623/1.11; 623/1.36

(57) ABSTRACT

A method and apparatus for repairing a vessel at a bifurcation, such as an aorto-ostium, without obstructing blood flow through the bifurcation. Delivery system having an anchor mechanism for positioning an expandable ostial stent within a diseased portion of a bifurcation so that the tubular body of the stent is seated within a side branch to the bifurcation, thereby repairing the vessel at the bifurcation without occluding blood flow. The anchor mechanism includes a plurality of wing-like members for holding the stent at a desired location in the side-branch of the main vessel. The stent delivery system may be used with a dilation catheter prior to deploying the anchor mechanism and stent.

### BACKGROUND OF THE INVENTION

The present invention relates to a stent delivery system configured for stent placement at a bifurcation of a patient's vasculature. In particular, the invention relates to a method and system for positioning and securing a stent at the aorto-ostium of an artery.

Several interventional treatment modalities are presently used for heart disease including by-pass surgery, balloon angioplasty and placement of stents in an occluded vasculature. By-pass surgery is still used for coronary applications by constructing a vascular detour around the occlusion. In typical balloon angioplasty procedures, a guiding catheter having a preformed distal tip is percutaneously introduced through the femoral artery into the cardiovascular system of a patient in a conventional Seldinger technique and advanced within the cardiovascular system until the distal tip of the guiding catheter is positioned at a desired location in a patient's vasculature, such as at an ostium. A guidewire is placed within an inner lumen of a dilatation (balloon) catheter, and then both are advanced to the distal portion of the guiding catheter. This technique is sometimes referred to as percutaneous transluminal coronary angioplasty (“PTCA”).

The distal portion of the guidewire is advanced out of the distal end of the guiding catheter into the patient's coronary vasculature until the distal end of the guidewire crosses a lesion to be dilated, then the dilatation catheter having an inflatable balloon on the distal portion thereof is advanced into the patient's coronary anatomy over the previously introduced guidewire until the balloon of the dilatation catheter is properly positioned across the lesion. Once in position across the lesion, the balloon, which is made of relatively inelastic materials, is inflated to a predetermined size with radiopaque liquid at relatively high pressure (for example, greater than four atmospheres) to compress the arteriosclerotic plaque of the lesion against the inside of the vessel wall and to otherwise expand the inner lumen of the vessel. The balloon is then deflated so that blood flow can be resumed through the dilated vessel and the dilatation catheter can be removed therefrom. Further details of dilatation catheters, guidewires, and devices associated therewith for angioplasty procedures can be found in U.S. Pat. No. 4,323,071 (Simpson-Robert); U.S. Pat. No. 4,439,185 (Lindquist); U.S. Pat. No. 4,516,972 (Samson); U.S. Pat. No. 4,538,622 (Samson, et al.); U.S. Pat. No. 4,554,929 (Samson, et al.); U.S. Pat. No. 4,616,652 (Simpson); U.S. Pat. No. 4,638,805 (Powell); U.S. Pat. No. 4,748,982 (Horzewski, et al.); U.S. Pat. No. 5,507,768 (Lau, et al.); U.S. Pat. No. 5,451,233 (Yock); and U.S. Pat. No. 5,458,651 (Klemm, et al.), which are hereby incorporated herein in their entirety by reference thereto.

PTCA is deficient in some patients due to recoil, scarring and/or proliferation of smooth muscle cells causing re-occlusion of the artery (called “restenosis”). To prevent abrupt closure and restenosis, stents were developed to provide structural support for maintaining an open vessel. Stent deployment in a vessel generally involves the introduction of a stent, in a contracted condition, into a vessel at the desired implantation or target site in the occluded vessel. The stent is expanded such that it is fixed in the desired position in apposition to the vessel wall. A balloon expandable stent may be fitted over a collapsed angioplasty balloon or other expandable portion of a stent delivery system, which is introduced into the vessel and inflated, thereby expanding the stent and deploying it in the desired location. Alternatively, self-expanding stents are configured to expand when released from the contracted condition.

Stents may be constructed of a metal or polymer and generally cylindrical in shape and hollow, are implanted within the vessel to maintain lumen size. The stent acts as a scaffold to support the lumen in an open position. Configurations of stents include a cylindrical sleeve defined by a mesh, interconnected stents, or like segments. Stent insertion may cause undesirable reactions such as inflammation, infection, thrombosis, and proliferation of cell growth that occludes the passageway. To assist in preventing these conditions, stents have been used with coatings to deliver drugs or other therapeutic agents at the site of the stent. Exemplary stents are disclosed in U.S. Pat. No. 5,292,331 (Boneau); U.S. Pat. No. 6,090,127 (Globerman); U.S. Pat. No. 5,133,732 (Wiktor); U.S. Pat. No. 4,739,762 (Palmaz) and U.S. Pat. No. 5,421,955 (Lau), which are hereby incorporated herein in their entirety by reference thereto.

The ostium of a vessel is located at the point where a side-branch vessel is in fluid communication with a larger parent vessel. For example, the aorta gives rise to the coronary arteries; the origin of each coronary artery as it branches from the aorta is referred to as an ostium. A lesion (for example, an atherosclerotic plaque) located at the ostium of a vessel is referred to as an “ostial lesion.” Stenting ostial lesions is often difficult due to precisely localizing the ostium during stent delivery and implantation, placement of the stent in the side-branch vessel without the stent significantly protruding into the parent vessel and maintaining proper position of the stent delivery system. To repair an ostial lesion, a stent is configured to cover the affected area without occluding blood flow in the adjoining vessel. When a stent is improperly positioned at an ostium of a vessel, it may extend into the adjoining vessel, thereby occluding blood flow to some degree. Furthermore, when the stent extends into the adjoining vessel, the stent may block access to portions of the adjoining vessel that require further intervention. As shown in FIGS. 1A and 1B, prior art stent delivery systems used for treating a side branch have resulted in improper placement of the stent. For example, the stent may be deployed in the side branch vessel so that a portion of the side branch vessel is not covered by the stent (FIG. 1A), or the stent is deployed such that a portion of the stent extends into the main vessel (FIG. 1B).

Accordingly there is a need for, and what was heretofore unavailable, a method and apparatus for maintaining proper position of the stent delivery system so as to deploy a stent in the side-branch vessel without the stent significantly protruding into the parent vessel. The present invention solves these and other needs.

### SUMMARY OF THE INVENTION

A method and apparatus for repairing a vessel at a bifurcation without obstructing blood flow through the bifurcation. The apparatus includes an ostial stent delivery system having an anchor mechanism for positioning the expandable ostial stent within a diseased portion of the bifurcation so that the tubular body of the stent is seated within a side branch to the bifurcation, thereby completely repairing the vessel at the bifurcation without occluding blood flow. The anchor mechanism includes a plurality of wing-like members for holding the stent at a desired location in the side-branch of the main vessel. The stent delivery system may be used for the placement of either balloon expandable or self-expanding stents in blood vessels or similar structures.

The present invention relates to a stent delivery system to be used in the placement of one or more stents at an ostial lesion in a side-branch of a patient's vasculature. The stent delivery system includes a catheter having an inflatable member configured at its distal portion, a stent disposed on the inflatable member, and an anchor mechanism positioned proximal of the inflatable member. The anchor mechanism is deployed using a sheath positioned over the proximal portion of the catheter. Prior to insertion of the stent delivery system into the vasculature, the anchor mechanism is configured in a contracted condition. When the distal portion of the stent delivery system is positioned proximate to the ostial lesion, the anchor mechanism is configured in an expanded condition by distal movement of the sheath. As the anchor mechanism is expanded it lodges against the wall of the parent vessel, thereby localizing the ostium of the side-branch vessel containing the lesion so as to ensure that the stent(s) is(are) in the proper position for deployment.

The present invention includes an apparatus for removably securing a catheter at an ostium of a vessel. The apparatus includes an inner catheter and an outer sheath slidably disposed over the inner catheter. The apparatus further includes an anchor mechanism configured with a plurality of expandable wings, wherein a proximal portion of the anchor mechanism is operably connected to the distal portion of the outer sheath and a distal portion of the anchor mechanism is secured to the distal portion of the inner catheter. Each wing of the anchor mechanism may be scored to about a thirty percent decrease in thickness or may include an actuator configured to bend each wing outwardly from the inner catheter as the outer sheath is moved in a distal direction relative to the inner catheter.

The present invention provides a stent delivery system configured with a stent catheter assembly and a sheath assembly slidably disposed over the stent catheter assembly. The stent delivery system also includes an anchor assembly having a plurality of bendable wings, wherein a proximal portion of the anchor assembly is operably connected to the distal portion of the sheath assembly and a distal portion of the anchor assembly is secured to the distal portion of the stent catheter assembly. The anchor assembly is configured to bend each wing outwardly from the stent catheter assembly as the sheath assembly is moved in a distal direction relative to the stent catheter assembly. The stent delivery system includes a stent disposed on an inflatable member of the stent catheter assembly, wherein the inflatable member is positioned distal of the anchor assembly. The proximal portion of the sheath assembly may be configured to secure the sheath assembly to the stent catheter assembly. The stent delivery system may further include a guidewire assembly, wherein the stent catheter assembly is configured with a lumen sized for slidably retaining a portion of the guidewire assembly. Alternatively, the stent delivery system may include a dilatation catheter assembly, wherein the stent catheter assembly is configured with a lumen sized for slidably retaining a portion of the dilatation catheter assembly. The dilatation catheter assembly may include a lumen for slidably retaining a guidewire.

The present invention includes a method for deploying a stent at the ostium of a vessel. The method includes providing a stent delivery system having an inner catheter and an outer sheath slidably disposed over the inner catheter. The stent delivery system further includes an anchor mechanism configured with a plurality of bendable wings, wherein a proximal portion of the anchor mechanism is operably connected to the distal portion of the outer sheath and a distal portion of the anchor mechanism is secured to the distal portion of the inner catheter, and wherein the anchor mechanism is configured to bend each wing outwardly from the inner catheter as the outer sheath is moved in a distal direction relative to the inner catheter. The stent delivery system also includes a stent disposed on an expandable member of the inner catheter, wherein the expandable member is positioned distal of the anchor mechanism.

The method of the present invention further includes introducing the stent delivery system into a vasculature of a patient, positioning the stent in a side-branch vessel of a main vessel in the vasculature so as to place the anchor mechanism proximate an ostium of the main vessel. The outer sheath is moved distally relative to the inner catheter so as to deploy the wings of the anchor mechanism. The expandable member of the inner catheter assembly is used to deploy the stent. The method also includes moving the outer sheath proximally relative to the inner catheter so as to straighten the wings of the anchor mechanism, contracting the expandable member of the inner catheter, and removing the stent delivery system from the vasculature of the patient so as to retain the stent in the side-branch vessel.

The aforementioned and other features and advantages of the invention will become further apparent from the following detailed description of the invention, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1A is an elevational view of a bifurcation in which a prior art stent is implanted in the side branch vessel.

FIG. 1B is an elevational view of a bifurcation in which a prior art stent is implanted in the side branch vessel, with the proximal end of the stent extending into the main vessel.

FIG. 2 is a side plan view in partial cross-section of an ostial stent delivery system in accordance with the present invention.

FIG. 3 is a perspective view of a stent in accordance with the present invention.

FIG. 4A is a side plan view of the distal portion of an ostial stent delivery system with the anchor mechanism in the deactivated configuration in accordance with the present invention.

FIG. 4B is a side plan view of the distal portion of an ostial stent delivery system with the anchor mechanism in a partially activated configuration in accordance with the present invention.

FIG. 4C is a side plan view of the distal portion of an ostial stent delivery system with the anchor mechanism in approximately a fully activated configuration in accordance with the present invention.

FIG. 5 is a flow diagram of one embodiment of a method for treating an ostium of a side-branch vessel, in accordance with the present invention.

FIG. 6 is a side plan view in partial cross section of the distal portion of an ostial stent delivery system of the present invention and a balloon catheter positioned at a target ostial lesion in a blood vessel.

FIG. 7 is a side plan view in partial cross section of the distal portion of an ostial stent delivery system of the present invention depicting pre-dilatation of an ostial lesion by inflation of a balloon catheter.

FIG. 8 is a side plan view in partial cross section of the distal portion of an ostial stent delivery system of the present invention depicting advancement of the deflated balloon catheter distal of the ostial lesion.

FIG. 9 is a side plan view in partial cross section of the distal portion of an ostial stent delivery system of the present invention depicting advancement of the stent proximate the ostial lesion.

FIG. 10 is a side plan view in partial cross section of the distal portion of an ostial stent delivery system of the present invention depicting partial distal advancement of the outer sheath and partial expansion of the anchor mechanism.

FIG. 11 is a side plan view in partial cross section of the distal portion of an ostial stent delivery system of the present invention depicting full distal advancement of the outer sheath and full expansion of the anchor mechanism.

FIG. 12 is a side plan view in partial cross section of the distal portion of an ostial stent delivery system of the present invention depicting expansion of the stent in a side branch of a blood vessel at the target ostial lesion.

FIG. 13 is a side plan view in partial cross section of the distal portion of an ostial stent delivery system of the present invention proximal advancement of the outer sheath and full contraction of the anchor mechanism prior to withdrawal of the stent delivery system from a blood vessel.

### DETAILED DESCRIPTION OF THE INVENTION

The method and apparatus of the present invention is configured for repairing a vessel at a bifurcation without obstructing blood flow through the bifurcation. Many other prior art attempts at implanting intravascular stents in a bifurcation have proved less than satisfactory. The present invention includes an assembly and method for treating bifurcations in, for example, at an aorto-ostium, in the coronary arteries and veins and in other vessels of a human body (patient). The apparatus of the present invention includes an ostial stent delivery system having an anchor mechanism for positioning an expandable ostial stent within a diseased portion of a bifurcation so that the tubular body of the stent is seated within a side branch to the bifurcation, thereby repairing the vessel at the bifurcation without occluding blood flow. The anchor mechanism includes a plurality of wing-like members for holding the stent at a desired location in the side-branch of the main vessel. The stent delivery system may be used for the placement of either balloon expandable or self-expanding stents in blood vessels or similar structures. In addition, the system may be used to deploy multiple stents in a single procedure, and may be used in conjunction with an anti-embolic filter.

Turning now to the drawings, in which like reference numerals represent like or corresponding aspects of the drawings, the stent delivery system may be configured as an over-the-wire type catheter system or a rapid exchange type catheter system for deploying a stent as generally described in U.S. Pat. Nos. 6,955,688; 6,616,689; 6,193,727; 5,514,154 and 4,323,071, which are hereby incorporated herein in their entirety by reference. In addition, a guiding catheter may be used having an internal diameter large enough to accommodate a guidewire, a balloon catheter and/or an ostial stent delivery system. For example, where a stent is to be placed in an ostial lesion of a coronary artery, a 6, 8, 9 or 10 French (F) external diameter guiding catheter and a guide wire having a 0.014 inch (0.036 cm) or 0.018 inch (0.046 cm) diameter and being 190 to 300 centimeters (cm) in length may be used.

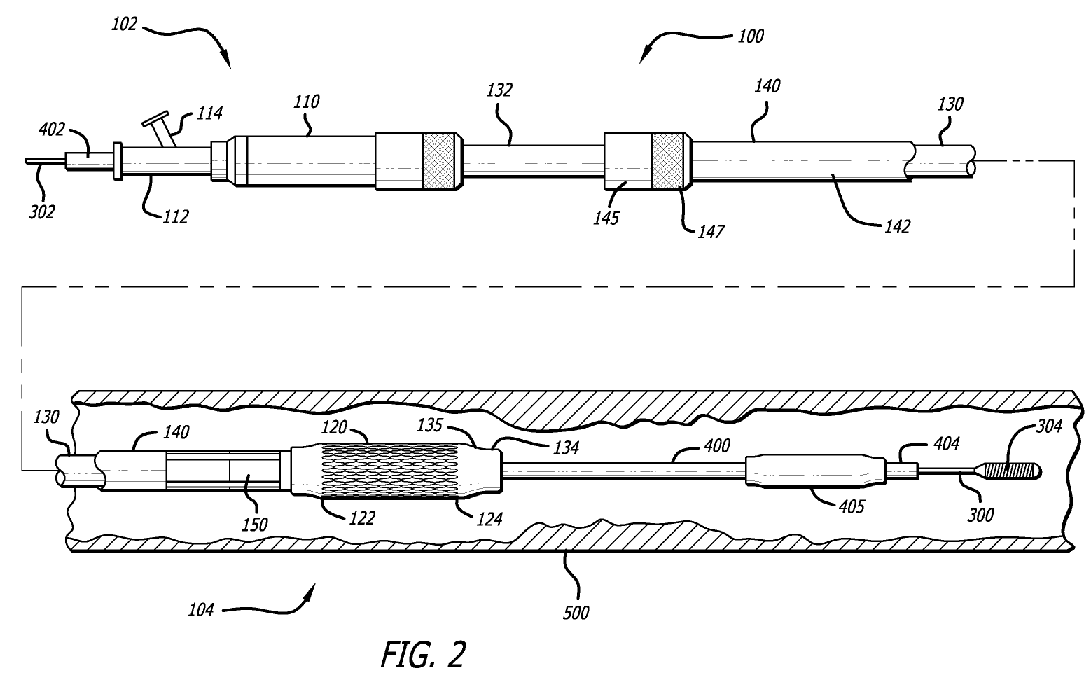

Referring now to FIG. 2, one embodiment of the stent delivery system 100 is an over-the-wire apparatus. The stent delivery system includes a proximal portion 102 and a distal portion 104. The stent delivery system includes a stent delivery catheter (inner catheter assembly) 130 having a distal expandable or inflatable portion such as a balloon 135 configured for carrying and deploying a stent or stents 120. The distal portion of the stent delivery system further includes an anchor mechanism 150 operably connected to an outer sheath 140 slidably disposed over the proximal and distal portions of the stent delivery catheter. The balloon may be positioned within two to three centimeters (cm) of the distal end 104 of the stent delivery catheter.

The proximal portion 102 of the stent delivery system, including the proximal portions of the stent delivery catheter 130 and the proximal portion of the outer sheath 140, is configured to reside outside of the patient so as to allow the operator to adjust the position of the proximal end 122 and distal end 124 of the stent 120 during placement within the vasculature 500 of a patient. A guiding dilatation catheter 400 having a dilatation balloon (inflatable or expandable member) 405 may be disposed within the stent delivery catheter and over the guidewire 300. The distal end 304 of the guidewire extends beyond the distal portion 404 of the dilatation catheter.

The proximal portion 102 of the stent delivery system 100 may be configured with a handle apparatus 110 secured to the proximal end 132 of the stent delivery catheter 130. The proximal end of the handle apparatus may be configured with an entry port 112 for slidably retaining the proximal portion 302 of the guidewire 300 and the proximal portion 402 of the dilatation catheter 400. The proximal portion of the stent delivery system may be further configured with a fitting 114 positioned proximal of the handle and configured to accept a syringe or other mechanism adapted to inflate the balloon.

For example, but not by way of limitation, the approximate longitudinal length of the stent delivery catheter 130 of the present invention for placement of an ostial stent 120 into an artery may be in the range from eighty to one-hundred eighty centimeters, for example about one-hundred fifty centimeters for the left main coronary ostium. The radial diameter of the shaft portion of the stent delivery catheter will depend upon whether it is configured to be placed over a guidewire and or dilatation catheter may be in the range of from 0.5 to 2.0 millimeters, for example, 0.6 millimeters when used over a guidewire alone and 1.6 millimeters when used with a dilatation catheter. The stent delivery catheter may be fabricated from a variety of suitable materials, including, but not limited to, polyethylene and nylon, polyamide, PEBAX, polytetrafluoroethylene (PTFE, TEFLON) or other biocompatible material. Such stent delivery systems may also be used to deliver a stent into other ostia originating from the aorta, including the renal arteries and brachio-cephalic arteries.

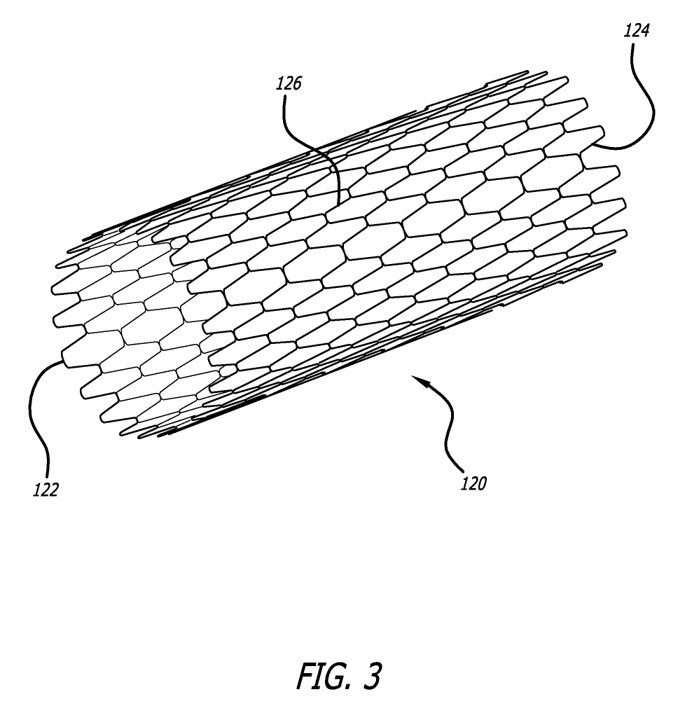

As depicted in FIG. 3, an ostial stent 120 is configured for deployment in main-vessel 500. The stent includes a proximal end 122 and a distal end 124 formed from a plurality of strut members 126. The stent may be made to be either balloon expandable or self-expanding. The stent can be formed from any of a number of materials including, but not limited to, stainless steel alloys, nickel-titanium alloys (the NiTi can be either shape memory or pseudoelastic), tantalum, tungsten, or any number of polymer materials. Such materials of manufacture are known in the art.

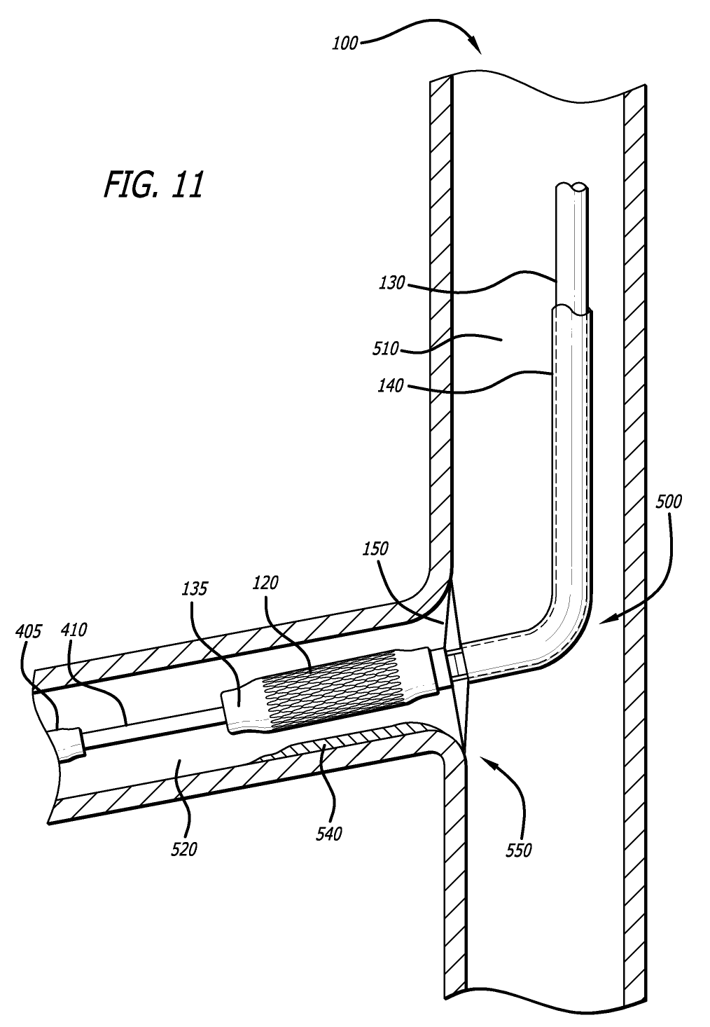

In one suitable embodiment, the ostial stent 120 is formed from a balloon-expandable, stainless steel material including a plurality of cylindrical elements connected by connecting members, wherein the cylindrical elements have an undulating or serpentine pattern. The stent, however, can have virtually any pattern suitable for treating an ostial lesion. The stent is mounted on a balloon portion (inflatable or expandable member) 135 of a catheter assembly 130 and crimped tightly onto the balloon to provide a low profile delivery diameter (see FIG. 2). After the catheter is positioned so that the stent and the balloon portion of the catheter are positioned at a desired location in the side-branch 520 from the main vessel 500 (see FIG. 11), the balloon is expanded, thereby expanding the stent beyond its elastic limit into contact with the vessel wall 530. Thereafter, the balloon is deflated and the balloon and catheter are withdrawn from the vessel, leaving the stent implanted.

The ostial stent delivery catheter 130 of the stent delivery system 100 may be deployed through the vasculature over a guidewire 300 and/or a balloon dilatation catheter 400, as shown in FIG. 2. The dilatation catheter may be fabricated from a variety of suitable materials, including, but not limited to, polyethylene and nylon, polyamide, polyether block amide (PEBAX), polytetrafluoroethylene (PTFE, TEFLON), polyetheretherketone (PEEK), polyolefins including high density polyethylene (HDPE) and polyethylene copolymers, polyimide, including blends and multilayers thereof or other biocompatible material. The length and radial diameter of the dilatation catheter may vary depending upon the vessel 500 or similar structure into which the stent 120 is to be placed. For example, but not by way of limitation, the approximate longitudinal length of the shaft of a dilatation catheter may be in the range of from eighty to one-hundred forty centimeters, for example from ninety to one-hundred twenty-five centimeters. The radial diameter of the shaft portion of the dilatation catheter may be in the range of from 0.8 to 1.6 millimeters, for example from 0.9 to 1.3 millimeters.

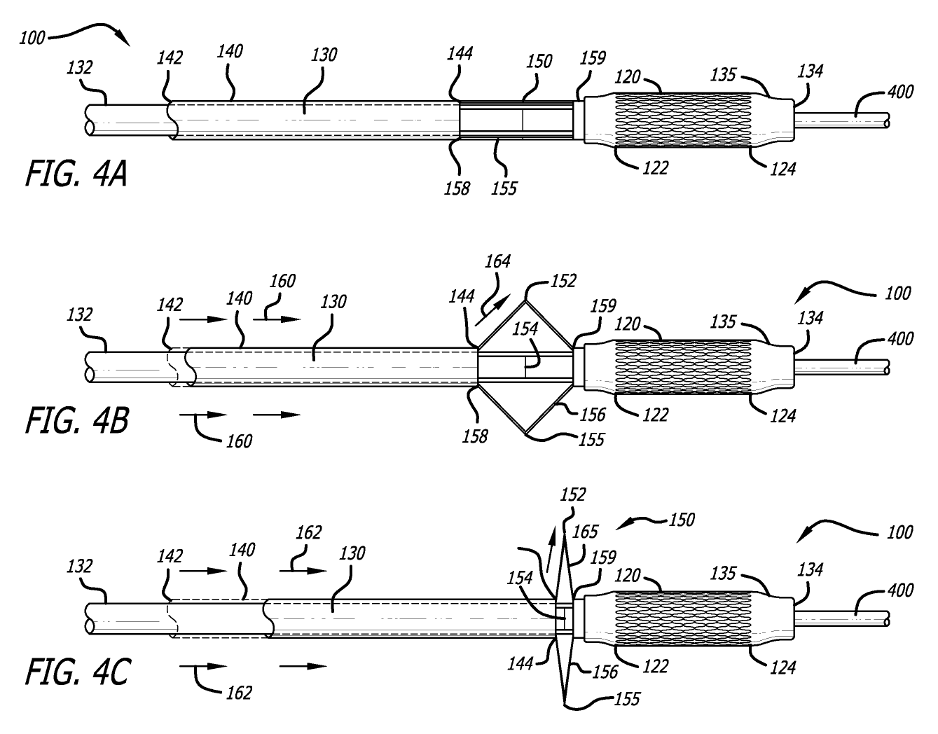

As further shown in FIGS. 4A, 4B, and 4C, one embodiment of the ostial lesion stent delivery system 100 includes a tubular guiding catheter 130 having a proximal portion 132 and a distal portion 134. The guiding catheter is configured from relatively torqueable and pushable materials useful for the placement of stents in blood vessel, including, but not limited to, polyethylene and nylon, polyamide, polyether block amide (PEBAX), polytetrafluoroethylene (PTFE, TEFLON), polyetheretherketone (PEEK), polyolefins including high density polyethylene (HDPE) and polyethylene copolymers, polyimide, including blends and multilayers thereof or other biocompatible material. The length and radial diameter of the guiding catheter may vary depending upon the vessel or similar structure into which the stent is to be placed. The outer radial diameter of the catheter may be in the range of from 1.0 to 2.0 millimeters, for example from 1.3 to 1.7 millimeters. The inner radial diameter may be in the range of from 0.8 to 1.6 millimeters, for example from 0.9 to 1.3 millimeters.

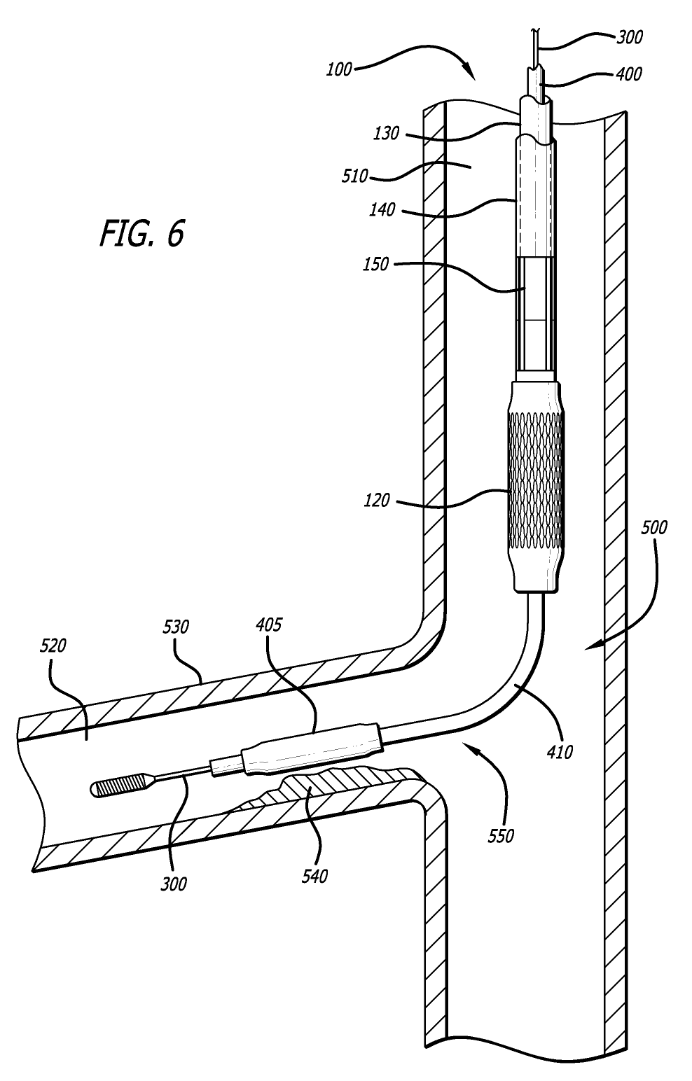

The stent delivery system 100 is configured to deliver a stent 120 or other implantable medical device proximate the ostium of a vessel (see FIG. 6). The stent may be configured from biocompatible polymers, metals and metal alloys, such as stainless steel, cobalt-chromium, nitinol or from other suitable implantable materials. The stent is mounted on the distal portion 134 of the guiding catheter 130 and is configured for expansion by an inflatable member such as a balloon 135. Alternatively, the stent may be self-expanding, which would require a sheath or other mechanism (not shown) to retain the stent until it is positioned at the desired deployment site in the vasculature 500. For conventional stents in use for treatment of coronary arteries, the length of an expandable portion (balloon) may be configured in the range of from five to thirty-five millimeters, for example from nine to thirty millimeters.

The stent delivery system 100 further includes an outer sheath 140 having a proximal portion 142 and a distal portion 144. The sheath is configured from a relatively stiff material formed from PTFE, polyolefins (for example, polyethylene, HDPE), polyesters (for example, PET), polyamides (for example, nylon, PEBAX), polyurethanes, polyvinyl chloride, polyimides or other suitable biocompatible materials. The stent delivery system is further configured with an anchor mechanism 150 having a plurality of wings 152, 154, 156 and 158. The anchoring mechanism includes a proximal portion 158 secured or otherwise fastened to the distal portion 144 of the outer sheath 140. The anchoring mechanism distal portion 159 is secured to the distal portion 134 of the guiding catheter 130 just proximal of the stent 120. Advancing the outer shaft 140 in a distal direction 160 (FIG. 3B) causes the wings of the anchor mechanism to move (deploy) in a perpendicular or radial direction 164 from the catheter shaft 130. As the outer shaft is moved in its most distal position 162, the anchoring mechanism wings become fully deployed in an almost perpendicular position 165 (FIG. 3C). The wings of the anchor mechanism are configured from a relatively flexible material such as polyolefins (for example, polyethylene, HDPE), polyesters (for example, PET), polyamides (for example, nylon, PEBAX), polyimides, polyurethanes, polyvinyl chloride or other suitable biocompatible materials.

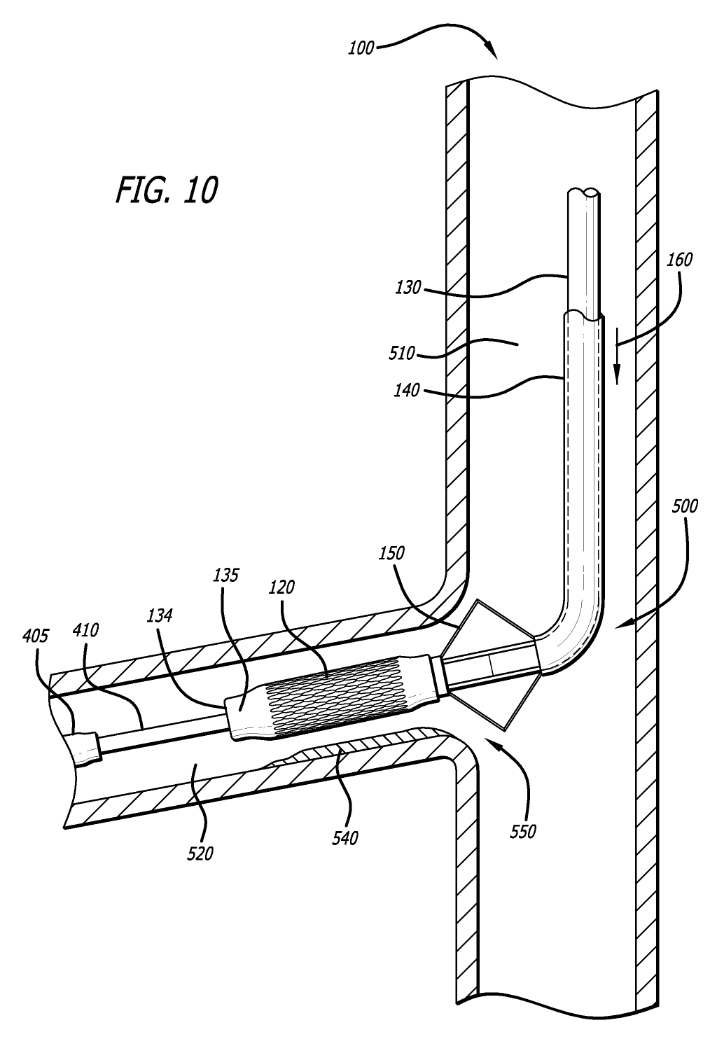

Accordingly, the distal portion 104 of the stent delivery system 100 may be positioned such that the anchoring mechanism wings 152, 154, 156 and 158 will abut the aorto-ostium 550 of an artery 500 and prevent forward motion of the stent delivery system (see FIG. 10). Since the wings are positioned at the proximal end of the stent, positioning of the anchoring mechanism 150 at the aorto-ostium will position the proximal edge 122 of the stent 120 at the ostium. The length of the anchor mechanism may be in the range from about five to twenty millimeters, for example about fifteen millimeters. The wings are configured with a mechanism 155 to permit the wings to bend in the middle or at another position as the outer sheath 140 is moved in the distal direction 160. The bending mechanism may be passive (for example, scoring to about a thirty percent decrease in thickness), or may be an active device (for example, an activated lever, fulcrum or other actuator). Markers (for example, radiopaque markers such as gold, tantalum or platinum bands or beads) may be placed at the proximal and/or distal ends of the anchor mechanism 158, 159 and/or on the expandable portion of the catheter 130 at the proximal and/or distal ends 122, 124 of the stent to aid in positioning the distal portion of the stent delivery system in the vasculature.

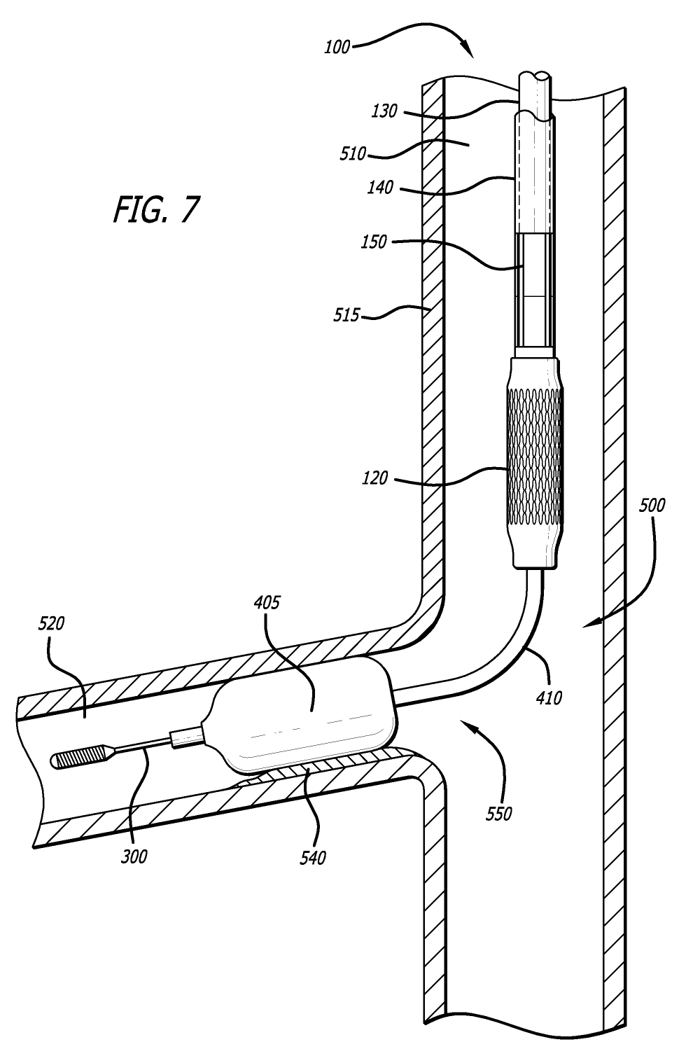

The balloon portion (inflatable or expandable member) 405 of the dilatation catheter 400 is configured to pre-dilate the ostial lesion 540 (see FIG. 7). For example, the balloon may be formed from a non-compliant high-pressure material having a collapsed diameter balloon smaller than or about the same diameter as the distal portion 304 of the balloon catheter. The balloon may be fabricated from polyethylene, nylon, polyamide copolymers such as PEBAX (polyether block amide), or polyurethanes including blends and multilayers thereof or other suitable biocompatible material. Where the balloon is to be used in coronary arteries, the balloon may be configured to have an inflated diameter ranging from two to five millimeters, for example from 2.5 to 4.5 millimeters, having an internal pressure of up to about twenty atmospheres, for example from four to twenty atmospheres. Such a balloon may be configured with a rated burst pressure of from twelve to twenty atmospheres.

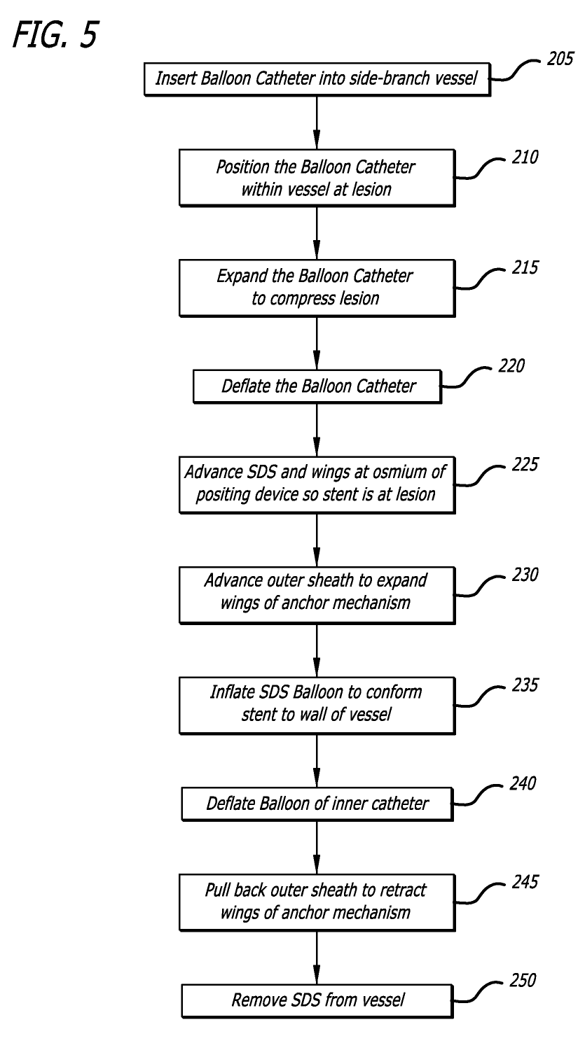

As shown in FIGS. 5-13, another aspect of the present invention are methods for treating an ostium of a side-branch vessel which include using one or more embodiments of the stent delivery system having an anchor mechanism. Various modifications to the method may be required depending on the structure into which the stent is to be placed, and the needs of particular patients as may be apparent to one having ordinary skill in the art. The method may be used for the placement of single or multiple self-expanding or non-self-expanding stents. The stent delivery system my be deployed into the vasculature using a guidewire in an over-the-wire configuration or in a rapid-exchange configuration.

Referring to FIG. 5, one embodiment of a method 200 in accordance with the present invention includes inserting (Block 205) the stent delivery system 100 over a balloon catheter 300 having its distal portion positioned in a side-branch vessel 520 of a patient's vasculature 500 at an ostium 550 of a main vessel 510. The stent delivery system includes an inner catheter 130, a stent 120 disposed on an inflatable portion 135 of the distal portion 134 of the inner catheter, an anchor mechanism positioned distal of the inflatable portion and an outer sheath slidably disposed on a proximal portion of the catheter and cowling to activate the anchor mechanism (FIGS. 4A, 4B and 4C).

As shown in FIG. 6, the side-branch vessel 520 or similar structure for repair may be identified, and a path for the ostial stent delivery system 100 may be established. In various embodiments, a guiding catheter and a guide wire may be inserted to provide the proper path. The remainder of this exemplary description relates to the use of a stent delivery system disposed over a dilatation catheter 400; however, the invention is not to be limited to such embodiments. Traction is maintained on the proximal portion 102 of the stent delivery system outside the patient (FIG. 2) so that the distal portion 404 of the dilatation catheter is positioned such that the balloon 405 is located at least partially within the side-branch vessel and over the ostial lesion 540. The distal portions 104, 134 of the stent delivery system are positioned adjacent the ostium 550 of the main vessel 510 (Block 210).

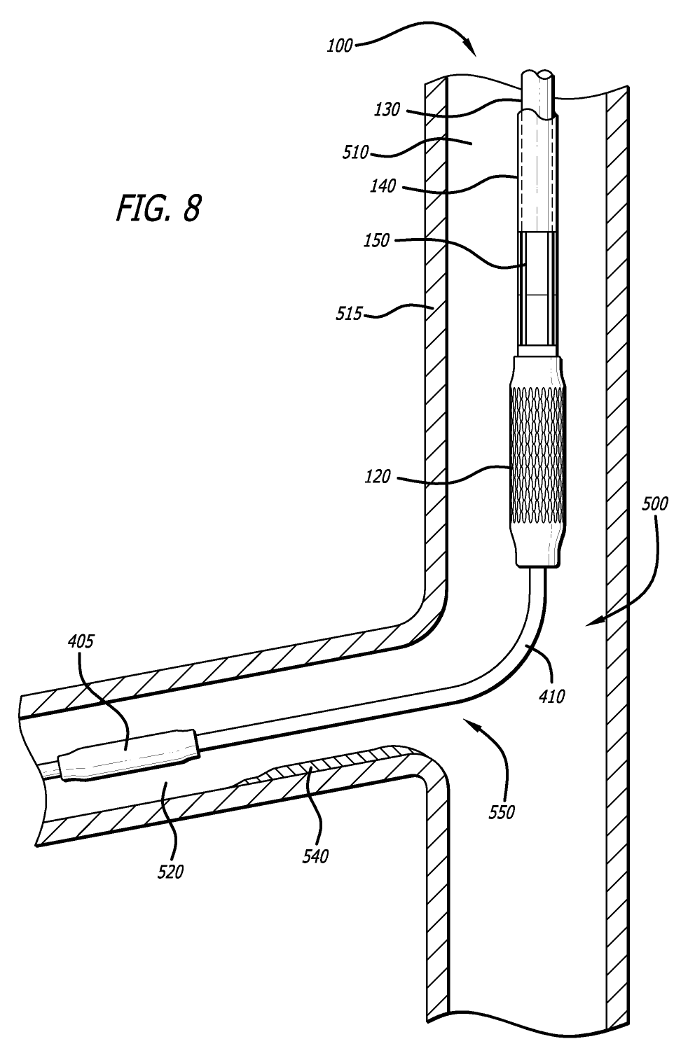

As shown in FIG. 7, the expandable portion 405 of the balloon catheter 400 may then be inflated to dilate the lesion within the side-branch vessel (Block 215). The balloon is then deflated (Block 220), and then advanced to a position distal to the ostial lesion 540, while the ostial stent delivery system distal portion 104 remains stationary in the main vessel 510 (see FIG. 8). When pre-dilatation may not be necessary, a guidewire may be used instead of the balloon catheter, or the balloon may be advanced distal to the ostial lesion without inflation.

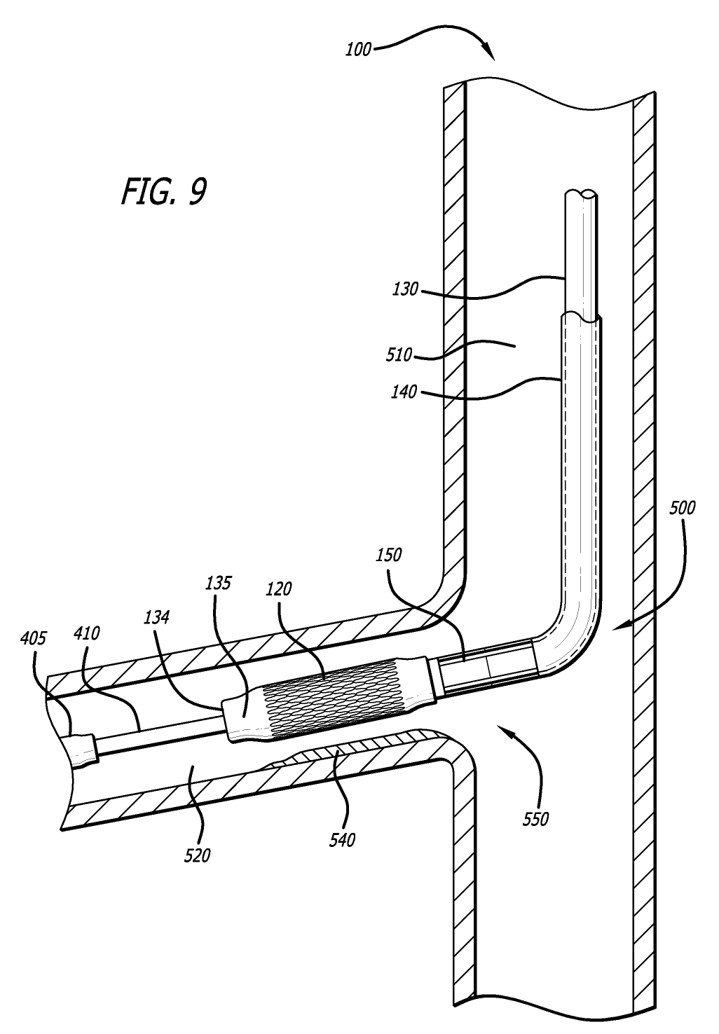

As shown in FIG. 9, The distal portion 104 of the ostial stent delivery system 100 may then be advanced into the side-branch vessel 520 over the shaft 410 of the balloon (dilatation) catheter 400 (Block 225). The ostial stent delivery system is advanced until the anchor mechanism 150 is positioned at the ostium 550 and adjacent the wall of the parent vessel 510 from which the target vessel branches (for example, at the wall of the aorta). A stent 120, removably fixed on the expandable portion 135 of the inner catheter 130, is thereby moved into the desired position over the ostial lesion 540. Radiopaque markers (not shown) defining the location of the stent may aid in stent positioning at the ostial lesion. Referring to FIG. 10, the outer sheath 140 is moved in a distal direction 160 relative to the inner catheter so as to expand the anchor mechanism wings 152, 154, 156, 158 (Block 230). Distal movement of the outer sheath causes the wings to move in an outward direction relative to the inner catheter at the bend or folds 155 of the anchor mechanism wings (see FIGS. 4A-4C).

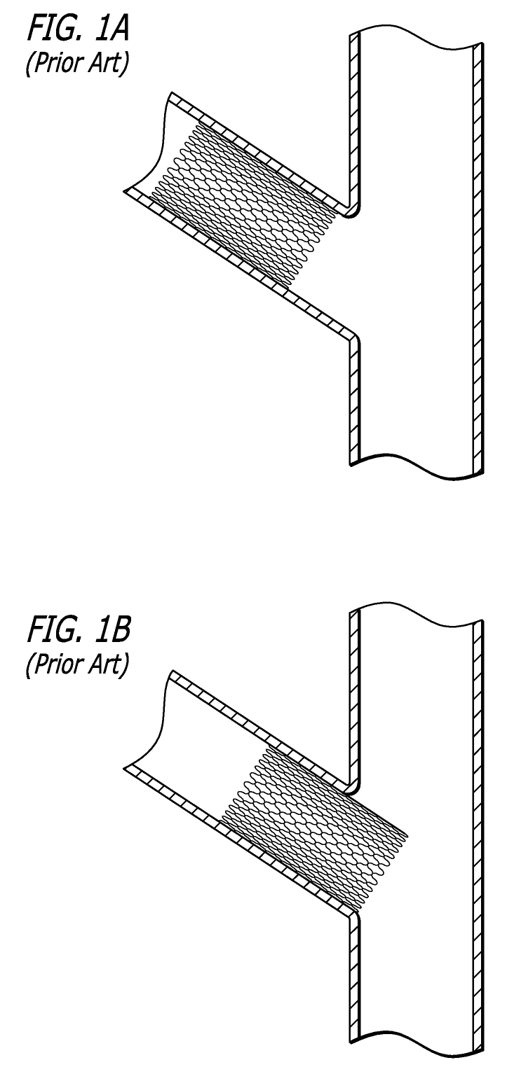

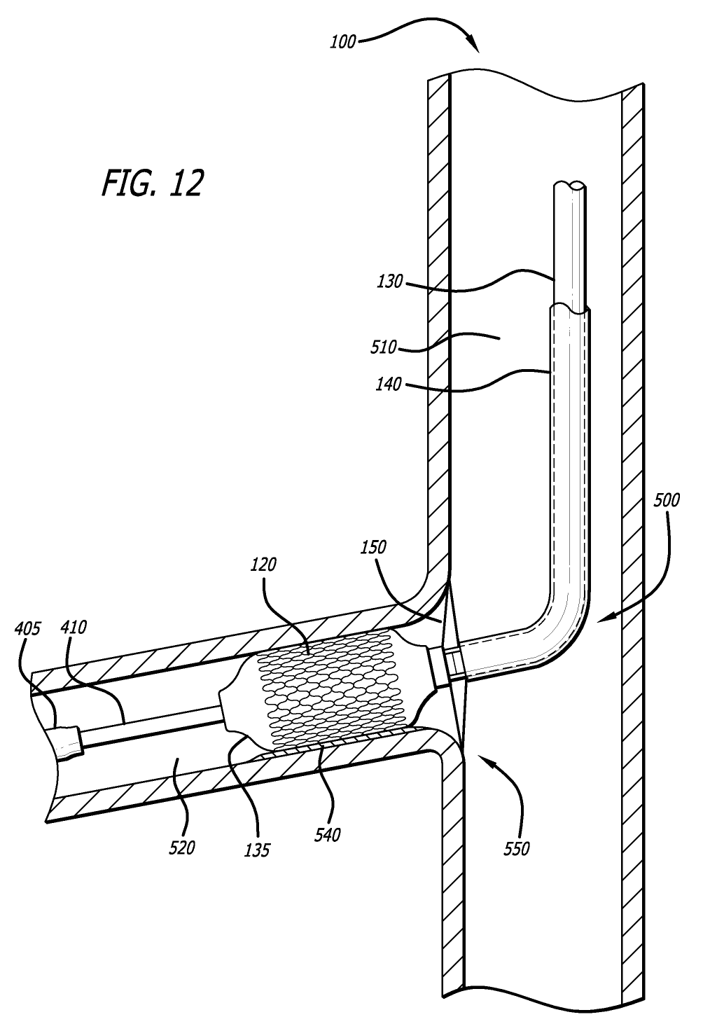

As shown in FIG. 1, the inner catheter 130 is anchored in the main vessel 510 by the anchor mechanism 150. Once the stent 120 is fixed in place, the inflatable member (balloon) 135 of the inner catheter is inflated (Block 235) to expand and anchor the stent within the side-branch vessel (FIG. 12). Where the stent includes a therapeutic agent (pharmaceutical substances), expanding the stent places the therapeutic agent in contact with the vessel wall. To aid in conforming the stent to the shape of the ostium, the stent may comprise a body portion and an end portion, these portions having different material properties or different geometric configurations. For example, the end portion may be made of a different, more malleable material than the body portion; or the end portion may have a geometric configuration that is more easily deformed than that of the body portion. Where a stent is a self-expanding stent, a separate mechanism, such as an inner sheath (not shown), may be used to deploy the stent.

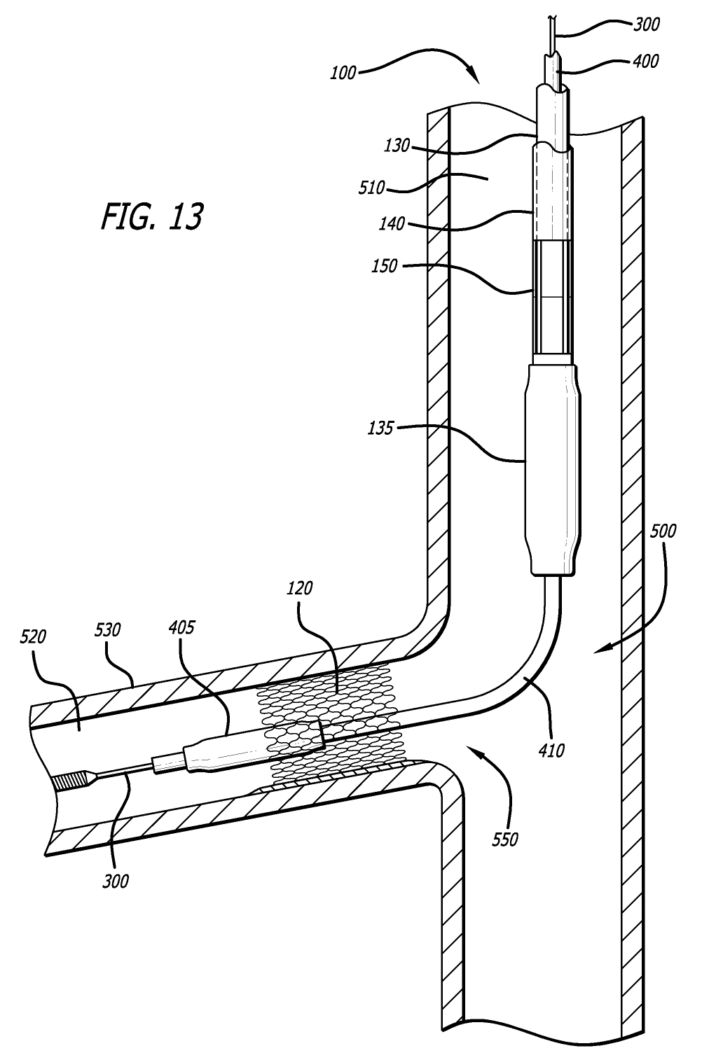

As shown in FIG. 13, after the stent 120 has been expanded at the ostial lesion 540 of the side-branch vessel 520, the inflatable member 135 of the inner catheter 130 is deflated (Block 240). The outer sheath 140 is then moved in a proximal direction 164 so as to collapse the wings of the anchor mechanism 150. The stent delivery system 110, including the inner catheter 130, outer sheath 140 and balloon catheter 300 and/or guidewire are removed from the side-branch vessel 520, the main vessel 510 and out of the patient's vasculature 500 (Block 250).

While particular forms of the present invention have been illustrated and described, it will also be apparent to those skilled in the art that various modifications can be made without departing from the spirit and scope of the invention. Accordingly, it is not intended that the invention be limited, except as by the appended claims.

I claim:

1. An apparatus for removably securing a catheter at an ostium of a vessel, comprising:
an inner catheter having a proximal portion and a distal portion;
an outer sheath having a proximal portion and a distal portion, wherein the outer sheath is slidably disposed over the inner catheter; and
an anchor mechanism configured with a plurality of expandable wings, wherein a proximal portion of the anchor mechanism is operably connected to the distal portion of the outer sheath and a distal portion of the anchor mechanism is secured to the distal portion of the inner catheter.

2. The apparatus of claim 1, wherein each wing of the anchor mechanism is scored to about a thirty percent decrease in thickness.

3. The apparatus of claim 1, wherein the anchor mechanism includes an actuator configured to bend each wing outwardly from the inner catheter as the outer sheath is moved in a distal direction relative to the inner catheter.

4. The apparatus of claim 1, wherein the anchor mechanism includes means for causing each wing to bend outwardly from the inner catheter as the outer sheath is moved in a distal direction relative to the inner catheter.

5. The apparatus of claim 1, wherein the wings of the anchor mechanism are configured from a biocompatible material consisting of polyolefins, polyesters, polyamides, polyurethanes, polyvinyl chloride and polyimides.

6. The apparatus of claim 1, wherein the wings of the anchor mechanism are configured from a biocompatible material consisting of polyethylene, HDPE, PET, nylon and PEBAX.

7. The apparatus of claim 1, wherein the proximal portion of the outer sheath includes a mechanism configured to secure the outer sheath to the inner catheter.

8. The apparatus of claim 7, wherein the distal portion of the inner catheter is configured with an inflatable member positioned distal of the anchor mechanism.

9. The apparatus of claim 8, wherein the inner catheter is configured with a first lumen configured for providing a fluid to expand the inflatable member.

10. The apparatus of claim 9, wherein the inner catheter is configured with a second lumen sized for slidably retaining a guidewire.

11. The apparatus of claim 9, wherein the inner catheter is configured with a second lumen sized for slidably retaining a dilatation catheter.

12. A stent delivery system, comprising:
a stent catheter assembly having a proximal portion and a distal portion;
a sheath assembly having a proximal portion and a distal portion, wherein the sheath assembly is slidably disposed over the stent catheter assembly;
an anchor assembly having a plurality of bendable wings, wherein a proximal portion of the anchor assembly is operably connected to the distal portion of the sheath assembly and a distal portion of the anchor assembly is secured to the distal portion of the stent catheter assembly, and wherein the anchor assembly is configured to bend each wing outwardly from the stent catheter assembly as the sheath assembly is moved in a distal direction relative to the stent catheter assembly; and
a stent disposed on an inflatable member of the stent catheter assembly, wherein the inflatable member is positioned distal of the anchor assembly.

13. The stent delivery system of claim 12, wherein the proximal portion of the sheath assembly is configured to secure the sheath assembly to the stent catheter assembly.

14. The stent delivery system of claim 13, wherein the stent catheter assembly is configured with a first lumen configured for providing a fluid to expand the inflatable member.

15. The stent delivery system of claim 14, further including a guidewire assembly, wherein the stent catheter assembly is configured with a second lumen sized for slidably retaining a portion of the guidewire assembly.

16. The stent delivery system of claim 14, further including a dilatation catheter assembly, wherein the stent catheter assembly is configured with a second lumen sized for slidably retaining a portion of the dilatation catheter assembly.

17. The stent delivery system of claim 16, further including a guidewire assembly, wherein the dilatation catheter assembly is configured with a third lumen sized for slidably retaining a portion of the guidewire assembly.

18. A method for deploying a stent at the ostium of a vessel, comprising:
(a) providing a stent delivery system, including,
an inner catheter having a proximal portion and a distal portion,
an outer sheath having a proximal portion and a distal portion, wherein the outer sheath is slidably disposed over the inner catheter,
an anchor mechanism configured with a plurality of bendable wings, wherein a proximal portion of the anchor mechanism is operably connected to the distal portion of the outer sheath and a distal portion of the anchor mechanism is secured to the distal portion of the inner catheter, and wherein the anchor mechanism is configured to bend each wing outwardly from the inner catheter as the outer sheath is moved in a distal direction relative to the inner catheter, and
a stent disposed on an expandable member of the inner catheter, wherein the expandable member is positioned distal of the anchor mechanism;
(b) introducing the stent delivery system into a vasculature of a patient;
(c) positioning the stent in a side-branch vessel of a main vessel in the vasculature so as to place the anchor mechanism proximate an ostium of the main vessel;
(d) moving the outer sheath distally relative to the inner catheter so as to deploy the wings of the anchor mechanism;
(e) expanding the expandable member of the inner catheter assembly so as to expand the stent;
(f) moving the outer sheath proximally relative to the inner catheter to as to contract the wings of the anchor mechanism;
(g) contracting the expandable member of the inner catheter; and
(h) removing the stent delivery system from the vasculature of the patient so as to retain the stent in the side-branch vessel.

19. The method of claim 16, wherein providing a stent delivery system further includes providing a guidewire, wherein the inner catheter is configured with a lumen sized for slidably retaining a portion of the guidewire, such that the guidewire is introduced into the vasculature prior to introducing the inner catheter and outer sheath over the guidewire.

20. The method of claim 16, wherein providing a stent delivery system further includes providing guidewire and a dilatation catheter, wherein the dilatation catheter is configured with a first lumen sized for slidably retaining a portion of the guidewire and wherein the inner catheter is configured with a second lumen sized for slidably retaining a portion of the dilatation catheter, such that the guidewire is introduced into the vasculature prior to introducing the dilatation catheter over the guidewire and the inner catheter and outer sheath are introduced over the dilatation catheter.
